(19) United States
(12) Patent Application Publication
(10) Pub. No.: US 2016/0244845 A1
(43) Pub. Date: Aug. 25, 2016

(54) METHOD FOR DETERMINING THE PROGNOSIS OF PANCREATIC CANCER

(71) Applicants: AB SCIENCE, Paris (FR); ACOBIOM, Grabels (FR)

(72) Inventors: David PIQUEMAL, SAINT CHRISTOL LES ALES (FR); Alain MOUSSY, PARIS (FR); Jean-Pierre KINET, Lexington, MA (US)

(73) Assignees: AB Science, Paris (FR); ACOBIOM, Grabels (FR)

(21) Appl. No.: 15/027,121

(22) PCT Filed: Oct. 3, 2014
(86) PCT No.: PCT/EP2014/071251

§ 371 Date: Apr. 4, 2016

(30) Foreign Application Priority Data
Oct. 4, 2013 (EP) 13306381.8
Oct. 4, 2013 (EP) PCT/EP2013/070741

(51) Int. Cl. C12Q 1/68

(52) U.S. Cl. CPC C12Q 1/6886; C12Q 1/686; C12Q 2600/158; C12Q 2600/106; C12Q 2600/118

(57) ABSTRACT

An in vitro method for determining the prognosis of pancreatic cancer in a patient includes the following steps: a) measuring the expression level of at least one gene chosen from the group consisting of: ACOX-1, TNFRSF10B, LYN, HIF1A, UBE2H, PARP2, ABCC1, ABCC3, IGJ and RPS23 or homologous genes, in a blood sample of the patient, b) predicting the outcome of the pancreatic cancer in the patient. a kit specifically designed to carry out such a method is also described.

The present invention relates to pancreatic cancer and more particularly to the prognosis of pancreatic cancer, especially of a pancreatic cancer treatment.

With 43,920 new diagnoses in the United States each year, and 37,390 deaths, mortality is over 85%, making pancreatic cancer the fourth highest cancer killer in the United States amongst both men and women.

The incidence of pancreatic cancer has markedly increased over the past several decades. Each year about 60,000 individuals in Europe, and more than 230,000 worldwide, are diagnosed with this condition.

Patients diagnosed with pancreatic cancer have often a poorer prognosis compared to other malignancies, in part because early detection is difficult. At the time of diagnosis, most patients with pancreatic ductal adenocarcinoma present with locally advanced or metastatic disease, and only 10-20% of cases are candidates for curative surgery. Median survival from diagnosis is around 3 to 6 months; 5-year survival is much less than 5% and complete remission is extremely rare.

Current therapies approved or used in clinical practice in pancreatic cancer patients are gemcitabine, folfirinox and erlotinib.

Gemcitabine is a nucleoside analog, often used in pancreatic cancer treatment. With gemcitabine, the median overall survival varies between 4.9 months and 8.3 months.

Folfirinox is a tritherapy that has shown to increase median overall survival to 11.1 months in a recent phase III study. However, after 2 years, no benefit in survival rates was detectable with folfirinox compared to treatment with gemcitabine alone. Furthermore, the additional toxicity related to folfirinox has negative impact on the treatment.

Erlotinib, the first tyrosine kinase inhibitor approved in combination treatment with gemcitabine, shows therapeutic benefit in terms of overall survival (OS) compared to gemcitabine treatment alone.

The limited treatment success and the continuing high mortality rate among pancreatic cancer patients highlight the high unmet medical need for additional therapeutic, well-tolerated products for this indication, ideally targeting different pathways implicated in the disease.

As an example of compounds targeting different pathways, erlotinib targets the human epidermal growth factor receptor type 1 (HER1 or EGFR), while other tyrorisine kinase inhibitors, such as Masitinib, potently and selectively inhibit the c-Kit wild-type (WT) receptor and several mutant forms of the same receptor.

The treatment of pancreatic cancer with different compounds may have different degrees of efficacy depending on the patient. However, up to today, there has been no means to predict the clinical benefit of the various available treatments. There is, thus, still a need for such prognosis tests in order to select the right treatment, so as to give the best chance to each patient. Said prognosis should be, in particular, a routinely performed test, such as a non-invasive test.

The inventors have identified a set of genes which can predict the outcome in pancreatic cancer, in particular, when a gemcitabine-based treatment is administered to the patient suffering from a pancreatic cancer. Said set of genes can be assessed directly from a blood sample.

The invention thus relates to an in vitro method for determining the prognosis of a pancreatic cancer in a patient, comprising the following steps:
a) Measuring the expression level of at least one gene or at least two genes chosen in the group consisting in ACOX-1, TNFRSF10B, LYN, HIF1A, UBE2H, PARP2, ABCC1, ABCC3, IGJ and RPS23 or homologous genes, in a blood sample of said patient;
b) Predicting the outcome of the pancreatic cancer in said patient.

The term “homologous” is defined as a polynucleotide sequence having a degree of identity of at least 80%, preferably 85%, more preferably 90%, and even more preferably 99% of the gene sequence (full length). The degree of identity refers to sequence identity between two sequences. Identity can be determined by comparing a position in each sequence which may be aligned for purposes of comparison. When an equivalent position in the compared sequences is occupied by the same base, then the molecules are identical at that position. Various alignment algorithms and/or programs may be used for determining the homology of two sequences, including FASTA and BLAST.

The method according to the invention is carried out on a blood sample of a patient, preferably on a whole peripheral blood sample of said patient. Peripheral blood is blood that circulates through the heart, arteries, capillaries and veins. The terms “whole blood” are used as opposed to a fraction of blood, obtained through separation of particular components of blood. An example of a blood fraction is peripheral blood mononuclear cells.

The method according to the invention is non-invasive because only a simple and routine blood sample collection is required to carry out the method. This is particularly advantageous since it is very difficult to access tumorous cells in pancreatic tissues for biopsy. Additionally, the sampling (collection, stabilization and transport) is standardized and the use of whole blood is safer than the use of a blood fraction such as peripheral blood mononuclear cells (PBNC), since it avoids handling errors related to the preparation of said fractions (for example FICOLL preparation for PBNC).

In a preferred embodiment, the expression level of at least 2, at least 3, at least 4, at least 5, at least 6, at least 7, at least 8, at least 9 and, more preferably of the 10 genes is measured.

By “prognosis”, it is meant the outcome of the patient in terms of life expectancy. In the case where the prognosis method involves a patient having or about to have a given pancreatic cancer treatment, the “outcome” results from the efficacy and/or the potential benefit of said given pancreatic cancer treatment, in particular, in terms of life expectancy.

Thus, the prognosis of pancreatic cancer, includes more particularly the prognosis of said cancer when a given pancreatic cancer treatment is administered to the patient. “Pancreatic cancer treatment” more specifically encompasses a gemcitabine-based treatment, more preferably, a treatment based on a combination of gemcitabine with a tyrosine kinase inhibitor, still more preferably, a treatment based on a combination of gemcitabine with masitinib.

Advantageously, the expression level of a gene is compared to a reference value, said value being, preferably, a reference expression level of said gene and, more preferably, the median or the first quartile expression level of said gene observed in patients suffering from a pancreatic cancer.

In particular, a modulated expression level of at least one or at least two of the above-mentioned genes, said expression level corresponding to either a lower expression level or a higher expression level depending upon the gene, will indicate survival of the patient depending upon the treatment received.

By “lower expression level”, it is meant an expression level that is lower by at least 5%, preferably 10%, than the mean expression level observed in patients suffering from a pancreatic cancer.

By “higher expression level”, it is meant an expression level that is higher by at least 5%, preferably 10%, than the mean expression level observed in patients suffering from a pancreatic cancer.

By “long-term survival”, it is understood survival for more than 10 months, preferably more than 12 months, even more preferably more than 15 months.

By “short-term survival”, it is meant a survival of less than 6 months, less than 5 months, or less than 3 months.

More precisely, a modulated expression level of at least one combination of genes selecting in the group consisting in:
ACOX1 and TNFRSF10B
RPS23 and ACOX1
ABCC3 and LYN
HIF1A and TNFRSF10
ABCC1 and IGJ
UBE2H and PARP2.

indicates survival of the patient depending upon the treatment received.

More precisely, these dual-gene combinations consist of: the concomitant up-regulation of genes ACOX-1 and TNFRSF10B; the concomitant down-regulation of gene RPS23 and up-regulation of gene ACOX-1; the concomitant up-regulation of genes ABCC3 and LYN; the concomitant up-regulation of genes HIF1A and TNFRSF10B; the concomitant down-regulation of genes ABCC1 and IGJ; the concomitant down-regulation of genes UBE2H and PARP-2.

In one embodiment, the invention relates to an in vitro method for determining the prognosis of a pancreatic cancer in a patient, comprising the following steps:
a) Measuring the expression level of at least ACOX-1 gene or homologous gene thereof, and optionally measuring the expression level of at least one or two of the following genes: TNFRSF10B, LYN, HIF1A, UBE2H, PARP2, ABCC1, ABCC3, IGJ and RPS23 or homologous genes thereof, in a blood sample of said patient;
b) Predicting the outcome of the pancreatic cancer in said patient.

The measurement of the gene expression level is performed by non-natural means. “Non-natural” means that such measurement does not occur in nature. In one embodiment, said measurement is performed by computer, computer-assisted tools or machine-assisted tools. Such computer and tools are known by a skilled person.

In another embodiment, the expression level of a gene is measured as the level of the protein of said gene. In that case, the level of the protein is preferably measured by employing antibody-based detection methods such as immunochemistry or western-blot analysis.

In another embodiment, the expression level of a gene is measured as the level of the RNA transcript or the cDNA of said genes. In that case, the level of RNA transcript(s) or the cDNA is measured by employing nucleic acid based detection methods such as microarrays, quantitative PCR, DNA chips, hybridization with labeled probes, or lateral flow immunoassays, in particular lateral flow dipstick tests.

Preferably, in the method according to the invention, the expression level of the gene is measured by real time quantitative PCR (real time quantitative polymerase chain reaction or qPCR) performed on the RNA transcript or the cDNA of said gene.

A real time quantitative PCR is a PCR wherein the amplified DNA is detected as the reaction progresses in real time. This detection is made through the accumulation of a fluorescent signal. The Ct (cycle threshold) is defined as the number of PCR cycles required for the fluorescent signal to cross the threshold (i.e. exceed background level).

Thus, a forward and a reverse primer, and a reporter, preferably a DNA fluorescent intercalant, are used in a qPCR. Advantageously, primers which are specific for hybridizing within the gene coding regions are used.

In the case of the ACOX1 gene, the primers amplify a sequence located on chromosome 17 between nucleotide 73,938,893 and nucleotide 73,939,007 (Assembly February 2009 GRch37/hg19, UCSC source).

In the case of the TNFRSF10B gene, the primers amplify a sequence located on chromosome 8 between nucleotide 22,877,657 and nucleotide 22,877,728 (Assembly February 2009 GRch37/hg19, UCSC source).

In the case of the RPS23 gene, the primers amplify a sequence located on chromosome 5 between nucleotide 81,571,951 and nucleotide 81,572,049 (Assembly February 2009 GRch37/hg19, UCSC source).

In the case of the ABCC3 gene, the primers amplify a sequence located on chromosome 17 between nucleotide 48,762,132 and nucleotide 48,762,221 (Assembly February 2009 GRch37/hg19, UCSC source).

In the case of the LYN gene, the primers amplify a sequence located on chromosome 8 between nucleotide 56,854,522 and nucleotide 56,860,210 (Assembly February 2009 GRch37/hg19, UCSC source).

In the case of the HIF1A gene, the primers amplify a sequence located on chromosome 14 between nucleotide 62,214,901 and nucleotide 62,214,976 (Assembly February 2009 GRch37/hg19, UCSC source).

In the case of the ABCC1 gene, the primers amplify a sequence located on chromosome 16 between the nucleotide 16,177,368 and nucleotide 16,180,772 (Assembly February 2009 GRch37/hg19, UCSC source).

In the case of the IGJ gene, the primers amplify a sequence located on chromosome 4 between the nucleotide 71,521,360 and nucleotide 71,521,432 (Assembly February 2009 GRch37/hg19, UCSC source).

In the case of the UBE2H gene, the primers amplify a sequence located on chromosome 7 between the nucleotide 129,470,836 and nucleotide 129,470,925 (Assembly February 2009 GRch37/hg19, UCSC source).

In the case of the PARP2 gene, the primers amplify a sequence located on chromosome 14 between the nucleotide 20,825,213 and nucleotide 20,825,283 (Assembly February 2009 GRch37/hg19, UCSC source).

In a preferred embodiment, the following primers can be used to perform the real time quantitative PCR:

ACOX1
primer forward:
(SEQ ID NO: 7)
TTTCTTCACTGCAGGGCTTT
primer reverse:
(SEQ ID NO: 8)
GGAAAGGAGGGATTTTGAGC
TNFRSF10B
primer forward:
(SEQ ID NO: 13)
GGTTTCATATTTAATTTGGTCATGG
primer reverse:
(SEQ ID NO: 14)
CAAACAAGGAAGCACATTGTGTA
RPS230
primer forward:
(SEQ ID NO: 15)
GATTTGGTCGCAAAGGTCAT
primer reverse:
(SEQ ID NO: 16)
TGCCTTTGTATAGGGCCAAA
ABCC1
primer forward:
(SEQ ID NO: 5)
CCAGTGGGGATCGGACAGA
primer reverse:
(SEQ ID NO: 6)
AGGGGATCATCGAAGAGGTAAAT
ABCC3
primer forward:
(SEQ ID NO: 17)
GGAGGACATTTGGTGGGCTTT
primer reverse:
(SEQ ID NO: 18)
CCCTCTGAGCACTGGAAGTC
LYN
primer forward:
(SEQ ID NO: 19)
ATCCAACGTCCAATAAACAGCA
primer reverse:
(SEQ ID NO: 20)
AAGGCTACCACAATGTCTCCT
HIF1A
primer forward:
(SEQ ID NO: 9)
TTTTGCTCTTTGTGGTTGGA
primer reverse:
(SEQ ID NO: 10)
CCTGGTCCACAGAAGATGTTT
IGJ
primer forward:
(SEQ ID NO: 11)
GGACATAACAGACTTGGAAGCA
primer reverse:
SEQ ID NO: 12)
TGGCAATTTCTTACACTAACCTGA
UBE2H
primer forward:
(SEQ ID NO: 23)
CGCAGGTTTTCCACTCATCT
primer reverse:
SEQ ID NO: 24)
ATGGCCATTTCTTCCCAAG
PARP2
primer forward:
(SEQ ID NO: 21)
GGGAAAGGAATCTACTTTGCTG
prime reverse:
(SEQ ID NO: 22)
TTCTTTAGGCGAGAGGCAAA

| | | Gene | Example of mRNA |
|---|---|---|---|
| | | identifiant | sequences |
| | | Sequence Id. | Sequence Id. |
| Name | Description | (Ensembl) | (Genbank) |
| ACOX1 | Acyl-CoA | ENSG00000161533 | NM_001185039.1 |
| | oxidase 1, | (SEQ ID NO 25) | (SEQ ID NO: 35) |
| | palmitoyl | | NM_004035.6 |
| | | | (SEQ ID NO: 36) |
| | | | NM_007292.5 |
| | | | (SEQ ID NO: 37) |
| TNFRSF10B | Tumor necrosis | ENSG00000120889 | NM_003842.4 |
| | factor receptor | (SEQ ID NO 26) | (SEQ ID NO: 38) |
| | superfamily, | | NM_147187.2 |
| | member 10b | | (SEQ ID NO: 39) |
| ABCC1 | ATP-binding | ENSG00000103222 | NM_004996.3 |
| | cassette, | (SEQ ID NO 27) | (SEQ ID NO: 40) |
| | sub-family C | | |
| | (CFTR/MRP), | | |
| | member 1 | | |
| ABCC3 | ATP-binding | ENSG00000108846 | NM_001144070.1 |
| | cassette, | (SEQ ID NO 28) | (SEQ ID NO: 41) |
| | sub-family C | | NM_003786.3 |
| | (CFTR/MRP), | | (SEQ ID NO: 42) |
| | member 3 | | |
| HIF1A | Hypoxia | ENSG00000100644 | NM_001243084.1 |
| | inducible | (SEQ ID NO 29) | (SEQ ID NO: 43) |
| | factor 1, | | NM_001530.3 |
| | alpha subunit | | (SEQ ID NO: 44) |
| LYN | V-yes-1 | ENSG00000254087 | NM_001111097.2 |
| | Yamaguchi | (SEQ ID NO 34) | (SEQ ID NO: 45) |
| | sarcoma viral | | NM_002350.3 |
| | related oncogene | | (SEQ ID NO: 46) |
| | homolog | | |
| IGJ | Immunoglobulin J | ENSG00000132465 | NM_144646.3 |
| | polypeptide, | (SEQ ID NO 30) | (SEQ ID NO: 47) |
| | linker protein | | |
| | for | | |
| | immunoglobulin | | |
| | alpha and | | |
| | mu polypeptides | | |
| UBE2H | Ubiquitin- | ENSG00000186591 | NM_001202498.1 |
| | conjugating | (SEQ ID NO 31) | (SEQ ID NO: 48) |
| | enzyme E2H | | NM_003344.3 |
| | | | (SEQ ID NO: 49) |
| PARP2 | Poly | ENSG00000129484 | NM_001042618.1 |
| | (ADP-ribose) | (SEQ ID NO 32) | (SEQ ID NO: 50) |
| | polymerase 2 | | NM_005484.3 |
| | | | (SEQ ID NO: 51) |
| RPS23 | Ribosomal | ENSG00000186468 | NM_001025.4 |
| | protein S23 | (SEQ ID NO 33) | (SEQ ID NO: 52) |
| GAPDH | glyceraldehyde- | ENSG00000111640 | NM_002046 |
| | 3-phosphate | | (SEQ ID NO: 53) |
| | dehydrogenase | | NM_001256799 |
| | | | (SEQ ID NO: 54) |
| B2M | beta-2 | ENSG00000166710 | NM_004048.2 |
| | microglobulin | | (SEQ ID NO: 55) |

The real time quantitative PCR allows one to determine the cycle threshold (Ct) value of gene, said value being normalized with respect to the expression level of a housekeeping gene to give a ΔCt value.

Housekeeping genes are genes that are expressed in all the cells of an organism under normal and pathophysiological conditions. These genes are usually expressed at relatively constant levels. Preferably, the normalization, in the method according to the invention, is based on the expression level of two housekeeping genes, in particular, based on the expression level of genes B2M and GAPDH.

In the case of the B2M gene, the amplified sequence is located on chromosome between nucleotides 45,010,919 and nucleotides 45,010,990 (Assembly February 2009 GRch37/hg19, UCSC source).

In the case of the GAPDH gene, the amplified sequence is located on chromosome 12 between nucleotides 6,643,999 and nucleotides 6,645,738 (Assembly February 2009 GRch37/hg19, UCSC source).

Primers particularly suitable for the GAPDH and B2M genes can be:

GAPDH
primer forward:
(SEQ ID NO: 1)
ATGGGGAAGGTGAAGGTCG
primer reverse:
(SEQ ID NO: 2)
GGGGTCATTGATGGCAACAATA
B2M
primer forward:
(SEQ ID NO: 3)
GCTCAGTAAAGACACAACCATCC
primer reverse:
(SEQ ID NO: 4)
CATCTGTGGATTCAGCAAACC

Thus, when two housekeeping genes (for example, genes B2M and GAPDH) are used to normalize the Ct value of a given gene, the ΔCt of said gene is calculated as follows:

ΔCt=Ct(gene)−[Ct(B2M)+Ct (GAPDH)]/2

Advantageously, for performing the real-time quantitative PCR, primers, size (preferably between 80 and 150 nucleotides), Tm (melting temperature, preferably 60° C.±1° C.), GC % (percentage of G or C nucleotide, preferably ˜60% in 3′), 3′ and 5′ self-complementarity and stability (preferably inferior to 4 nucleotides), product size ranges and thermodynamic parameters (secondary structure evolution according primer Tm and sodium salt concentration) are selected to allow a simultaneous detection.

According to the method of the invention, a patient presenting at least one of the six following features is predicted to have a short-term survival if treated with gemcitabine as a single agent, and is therefore eligible for a combination-based gemcitabine treatment, more particularly a gemcitabine+masitinib treatment:
a ΔCt value for ACOX1<=3.05
a ΔCt value for ACOX1<=3.05 and TNFRSF10B<=6,
a ΔCt value for RPS23>0.35 and ACOX1<=3.05,
a ΔCt value for ABCC3<=4.3 and LYN<=1.65,
a ΔCt value for HIF1A<=3.95 and TNFRSF10<=5.65,
a ΔCt value for ABCC1>3.5 and IGJ>7.05,
a ΔCt value for UBE2H>3.7 and PARP2>7.1,

A contrario, a patient presenting with none of the six aforementioned features is predicted to have a long-term survival if treated with gemcitabine as a single agent.

The present invention further relates to a nucleic acid microarray having on its surface nucleic acids consisting of nucleic acids able to hybridize with at least one combination of genes selected in the group consisting of:
ACOX1 and TNFRSF10B
RPS23 and ACOX1
ABCC3 and LYN
HIF1A and TNFRSF10
ABCC1 and IGJ
UBE2H and PARP2.
   with optionally nucleic acids specific for at least one housekeeping gene, preferably two housekeeping genes, more preferably for B2M and GAPDH.

The present invention also relates to a kit for determining the prognosis of pancreatic cancer in a patient, comprising means for detecting the level of expression of at least two genes selected from the group consisting in ACOX-1, TNFRSF10B, LYN, HIF1A, UBE2H, PARP2, ABCC1, ABCC3, IGJ and RPS23.

The means for detecting the level of expression can be a microarray according to the invention, a set of primers and a reporter such as fluorescent agents, labeled hydrolysis probes, molecular beacons, hybridization probes, chips and antibodies.

Preferably, the kit according to the invention comprises means for detecting the expression level of a combination of genes selecting in the group consisting in:
ACOX1 and TNFRSF10B
RPS23 and ACOX1
ABCC3 and LYN
HIF1A and TNFRSF10
ABCC1 and IGJ
UBE2H and PARP2.

More preferably, the kit according to the invention comprises means for detecting all the above-mentioned gene combinations.

The kit can further comprise instructions for use in the in vitro method according to the invention.

Finally, the invention also concerns the use of at least two genes selected in the group consisting in ACOX-1, TNFRSF10B, LYN, HIF1A, UBE2H, PARP2, ABCC1, ABCC3, IGJ and RPS23 for the prognosis of pancreatic cancer, in particular, of a pancreatic cancer treatment.

Preferably, the invention relates to the use of at least one of the combinations of genes selected in the group consisting in:
ACOX1 and TNFRSF10B,
RPS23 and ACOX1,
ABCC3 and LYN,
HIF1A and TNFRSF10B,
ABCC1 and IGJ,
UBE2H and PARP2,
   for said prognosis.

### EXAMPLE 1

### Set of Genes for the Prognosis of Pancreatic Cancer

1. Total blood samples from patients in PAXgene tubes in ice dry (shipper: LabConnect, USA) were received and stored at −80° C.
Collected tubes belong to 119 patients before treatment, and are named Week 0.
Total RNA was extracted from the blood samples of 119 patients before treatment, and named week 0. The transcriptome analysis (biomarker investigation) was conducted only on this time point.
All of the 119 RNA samples were analyzed. If some samples received were not eligible for analysis due to insufficient quality material, they were not used.
Digital Gene Expression (DGE) experiments were carried out to select a set of putative biomarkers.
Biomarker validation was done using Real-Time PCR on COBAS platform (LC480, ROCHE Diagnostics) and appropriate biostatistical approaches has been used to filter best biomarkers.

### 2. RNA Samples

119 blood RNA samples, corresponding to baseline blood samples, were extracted from blood (PAXgene Blood collection tubes, BD) using PAXgene Blood RNA Kit V.2 (PreAnalitix) according to manufacturer's recommendations.

| Subject Identifier | | | OS | OS |
|---|---|---|---|---|
| for the Study | Treatment group | Dead | (days) | (months) |
| 109 | Masitinib + Gemcitabine | YES | 182 | 6.0 |
| 110 | Placebo + Gemcitabine | YES | 183 | 6.0 |
| 111 | Placebo + Gemcitabine | NO | 744 | 24.4 |
| 112 | Placebo + Gemcitabine | YES | 112 | 3.7 |
| 113 | Placebo + Gemcitabine | NO | 589 | 19.4 |
| 207 | Placebo + Gemcitabine | YES | 98 | 3.2 |
| 208 | Placebo + Gemcitabine | YES | 87 | 2.9 |
| 209 | Masitinib + Gemcitabine | YES | 60 | 2.0 |
| 211 | Placebo + Gemcitabine | YES | 160 | 5.3 |
| 506 | Masitinib + Gemcitabine | YES | 147 | 4.8 |
| 507 | Masitinib + Gemcitabine | YES | 92 | 3.0 |
| 508 | Placebo + Gemcitabine | YES | 253 | 8.3 |
| 709 | Masitinib + Gemcitabine | YES | 474 | 15.6 |
| 710 | Masitinib + Gemcitabine | YES | 536 | 17.6 |
| 805 | Placebo + Gemcitabine | YES | 654 | 21.5 |
| 806 | Masitinib + Gemcitabine | YES | 167 | 5.5 |
| 1103 | Masitinib + Gemcitabine | YES | 449 | 14.8 |
| 1104 | Placebo + Gemcitabine | YES | 402 | 13.2 |
| 1203 | Placebo + Gemcitabine | YES | 252 | 8.3 |
| 1204 | Masitinib + Gemcitabine | YES | 436 | 14.3 |
| 1408 | Masitinib + Gemcitabine | YES | 432 | 14.2 |
| 1409 | Masitinib + Gemcitabine | YES | 49 | 1.6 |
| 1501 | Masitinib + Gemcitabine | YES | 47 | 1.5 |
| 1502 | Masitinib + Gemcitabine | YES | 560 | 18.4 |
| 1503 | Masitinib + Gemcitabine | YES | 519 | 17.1 |
| 1609 | Masitinib + Gemcitabine | YES | 498 | 16.4 |
| 1610 | Masitinib + Gemcitabine | YES | 492 | 16.2 |
| 1611 | Masitinib + Gemcitabine | YES | 188 | 6.2 |
| 1612 | Placebo + Gemcitabine | YES | 47 | 1.5 |
| 1613 | Placebo + Gemcitabine | YES | 73 | 2.4 |
| 1614 | Masitinib + Gemcitabine | YES | 312 | 10.3 |
| 1903 | Masitinib + Gemcitabine | YES | 355 | 11.7 |
| 2008 | Masitinib + Gemcitabine | YES | 235 | 7.7 |
| 2009 | Placebo + Gemcitabine | YES | 113 | 3.7 |
| 2403 | Placebo + Gemcitabine | YES | 222 | 7.3 |
| 2703 | Placebo + Gemcitabine | YES | 61 | 2.0 |
| 2704 | Placebo + Gemcitabine | YES | 134 | 4.4 |
| 3107 | Masitinib + Gemcitabine | YES | 483 | 15.9 |
| 3108 | Masitinib + Gemcitabine | YES | 376 | 12.4 |
| 3109 | Masitinib + Gemcitabine | YES | 349 | 11.5 |
| 3110 | Placebo + Gemcitabine | YES | 260 | 8.5 |
| 3111 | Placebo + Gemcitabine | YES | 144 | 4.7 |
| 3112 | Masitinib + Gemcitabine | YES | 112 | 3.7 |
| 3308 | Placebo + Gemcitabine | YES | 217 | 7.1 |
| 3309 | Placebo + Gemcitabine | YES | 112 | 3.7 |
| 3406 | Masitinib + Gemcitabine | YES | 104 | 3.4 |
| 3407 | Placebo + Gemcitabine | YES | 171 | 5.6 |
| 3408 | Placebo + Gemcitabine | YES | 350 | 11.5 |
| 3409 | Masitinib + Gemcitabine | YES | 136 | 4.5 |
| 3706 | Placebo + Gemcitabine | NO | 774 | 25.4 |
| 4407 | Placebo + Gemcitabine | YES | 135 | 4.4 |
| 4408 | Masitinib + Gemcitabine | YES | 96 | 3.2 |
| 4409 | Placebo + Gemcitabine | YES | 515 | 16.9 |
| 4410 | Placebo + Gemcitabine | NO | 708 | 23.3 |
| 4411 | Placebo + Gemcitabine | YES | 105 | 3.4 |
| 4412 | Masitinib + Gemcitabine | YES | 194 | 6.4 |
| 4413 | Masitinib + Gemcitabine | YES | 186 | 6.1 |
| 4414 | Placebo + Gemcitabine | YES | 437 | 14.4 |
| 4415 | Placebo + Gemcitabine | YES | 17 | 0.6 |
| 4416 | Masitinib + Gemcitabine | YES | 226 | 7.4 |
| 4503 | Placebo + Gemcitabine | NO | 700 | 23.0 |
| 4702 | Placebo + Gemcitabine | YES | 31 | 1.0 |
| 4703 | Masitinib + Gemcitabine | YES | 141 | 4.6 |
| 4801 | Masitinib + Gemcitabine | YES | 136 | 4.5 |
| 4802 | Masitinib + Gemcitabine | YES | 128 | 4.2 |
| 4803 | Masitinib + Gemcitabine | YES | 258 | 8.5 |
| 4902 | Placebo + Gemcitabine | YES | 161 | 5.3 |
| 4903 | Placebo + Gemcitabine | NO | 602 | 19.8 |
| 5006 | Masitinib + Gemcitabine | YES | 256 | 8.4 |
| 5008 | Placebo + Gemcitabine | YES | 588 | 19.3 |
| 5201 | Placebo + Gemcitabine | YES | 584 | 19.2 |
| 5202 | Placebo + Gemcitabine | YES | 43 | 1.4 |
| 5331 | Placebo + Gemcitabine | YES | 699 | 23.0 |
| 5332 | Masitinib + Gemcitabine | YES | 517 | 17.0 |
| 5333 | Masitinib + Gemcitabine | NO | 128 | 4.2 |
| 5334 | Masitinib + Gemcitabine | YES | 131 | 4.3 |
| 5335 | Masitinib + Gemcitabine | YES | 740 | 24.3 |
| 5336 | Placebo + Gemcitabine | YES | 486 | 16.0 |
| 5337 | Masitinib + Gemcitabine | YES | 265 | 8.7 |
| 5339 | Placebo + Gemcitabine | YES | 65 | 2.1 |
| 5340 | Placebo + Gemcitabine | YES | 356 | 11.7 |
| 5341 | Placebo + Gemcitabine | YES | 120 | 3.9 |
| 5342 | Placebo + Gemcitabine | YES | 393 | 12.9 |
| 5343 | Masitinib + Gemcitabine | YES | 107 | 3.5 |
| 5344 | Placebo + Gemcitabine | YES | 667 | 21.9 |
| 5345 | Placebo + Gemcitabine | YES | 251 | 8.2 |
| 5346 | Placebo + Gemcitabine | YES | 163 | 5.4 |
| 5501 | Masitinib + Gemcitabine | YES | 57 | 1.9 |
| 5602 | Masitinib + Gemcitabine | YES | 173 | 5.7 |
| 5702 | Masitinib + Gemcitabine | YES | 115 | 3.8 |
| 5703 | Placebo + Gemcitabine | YES | 261 | 8.6 |
| 5704 | Masitinib + Gemcitabine | NO | 744 | 24.4 |
| 5705 | Masitinib + Gemcitabine | YES | 254 | 8.3 |
| 5901 | Placebo + Gemcitabine | YES | 555 | 18.2 |
| 6201 | Placebo + Gemcitabine | YES | 52 | 1.7 |
| 6301 | Masitinib + Gemcitabine | YES | 341 | 11.2 |
| 6302 | Masitinib + Gemcitabine | YES | 408 | 13.4 |
| 6303 | Placebo + Gemcitabine | YES | 269 | 8.8 |
| 8001 | Placebo + Gemcitabine | YES | 458 | 15.0 |
| 8002 | Masitinib + Gemcitabine | YES | 347 | 11.4 |
| 8003 | Placebo + Gemcitabine | YES | 335 | 11.0 |
| 8106 | Placebo + Gemcitabine | YES | 461 | 15.1 |
| 8107 | Masitinib + Gemcitabine | YES | 373 | 12.3 |
| 8109 | Masitinib + Gemcitabine | YES | 195 | 6.4 |
| 8201 | Masitinib + Gemcitabine | YES | 305 | 10.0 |
| 8501 | Masitinib + Gemcitabine | YES | 216 | 7.1 |
| 8502 | Masitinib + Gemcitabine | YES | 144 | 4.7 |
| 8901 | Placebo + Gemcitabine | YES | 460 | 15.1 |
| 9311 | Masitinib + Gemcitabine | NO | 590 | 19.4 |
| 9312 | Placebo + Gemcitabine | YES | 141 | 4.6 |
| 9508 | Placebo + Gemcitabine | YES | 169 | 5.6 |
| 9509 | Placebo + Gemcitabine | YES | 318 | 10.4 |
| 9901 | Placebo + Gemcitabine | YES | 153 | 5.0 |
| 9903 | Masitinib + Gemcitabine | YES | 181 | 5.9 |
| 10303 | Placebo + Gemcitabine | YES | 131 | 4.3 |
| 10304 | Placebo + Gemcitabine | YES | 234 | 7.7 |
| 10305 | Masitinib + Gemcitabine | YES | 480 | 15.8 |
| 10306 | Masitinib + Gemcitabine | YES | 295 | 9.7 |
| 11001 | Masitinib + Gemcitabine | YES | 57 | 1.9 |
| 11205 | Masitinib + Gemcitabine | YES | 168 | 5.5 |
| 11207 | Placebo + Gemcitabine | YES | 231 | 7.6 |

Control of RNA integrity was performed with the 2100 Bioanalyzer (Agilent Technologies, Palo Alto, USA) using Eukaryotic Total RNA 6000 Nano Chip (Agilent Technologies). RNA quantity was controlled using NanoDrop ND-1000 spectrophotometer. Purified RNAs were conserved at −80° C.

### 3. DGE Library Construction and Tag-to-Gene Mapping

Twelve Digital Gene Expression (DGE) libraries were constructed from pooled blood RNA samples of patients. For each of the four treatment groups (i.e. Placebo/Gemcitabine P or Masitinib+Gemcitabine M & dead before month 4, M4, or alive after month 15, M15), three DGE libraries were constructed using the same pooled blood RNA samples (three technical replicates). The libraries were constructed with Illumina's DGE Tag Profiling kit according to the manufacturer's protocol (version 2.1B), using 2 μg of total RNA (equimolar amounts of RNA in the pool between each RNA sample). Sequencing analysis and base calling were carried out using the Illumina Pipeline, and sequence tags were obtained after purity filtering. The platform used was MGX (Montpellier, France). Data from each DGE library were analyzed with BIOTAG software (Skuldtech, Montpellier, France) for tag detection, tag counting and for assessing DGE library quality (Piquemal et al., 2002).

### 4. Taq Annotation and Selection

A local database compiling homo sapiens sequences and related information from well-annotated sequences of UniGene clusters (Built#232, March 2012, NCBI) was generated. For each sequence of this database, the expected DGE tag (canonical tag) located upstream the 3′-nearest NIaIII restriction site (CATG) of the sequence (R1), as well as putative tags located in inner positions (labeled as R2, R3 and R4 starting from the 3′ end of the transcript), were extracted (Piquemal et al., 2002). Experimental tags obtained from DGE libraries were matched and annotated (exact matches for the 17 bp) using this collection of virtual tags. Firstly, a correspondence for each experimental tag with the virtual canonical tags (R1) was looked for. Then, unmatched experimental tags with the R2 tags, then with R3, and R4 were annotated.

The analyses of the DGE experiments were carried out using edgeR Method (version 2.6.9, Bioconductor). The analyzed genes were selected according to (1) mathematic filters with the highest differential Fold Change (>1.5), FDR (False Discovery Rate) adjusted p-value criterion (<10%) based on the type I (α=5%) error reported in General considerations and (2) biologic filters with involvement of targeted genes in specific processes and known metabolic pathways.

5. cDNA Synthesis for Real-Time PCR

Reverse transcriptions were carried out for each of the 119 RNA in 20 μl final reaction volume with 300ng of total RNA using 200 units of SuperScript II enzyme (M-MLV RT Type, Invitrogen) and 250 ng of random primers according to manufacturer's instructions (25° C. 10 min, 42° C. 50 min, 70° C. 15 min the same day with the same pipettor set and the same manipulator.

### 6. Real-Time PCR

The validation of targeted genes was carried out on Real-Time PCR (qPCR) platform from Roche Diagnostics.

The qPCR experiments were carried out using LightCycler® 1536 DNA Green Master Kit and RealTime ready DNA Probes Master Kit (Roche Diagnostics) on Roche Diagnostics LightCycler1536® qPCR apparatus according to manufacturer's instructions.

For Sybr Green assays, the reaction mixture was prepared in a final volume of 2 μl as follows: 0,4 μl of LightCycler 1536 DNA Green Master 5× (Roche), 0,1 μl of Bright Green 20× (Roche), 0,1 μl of Setup Control 20× (Roche), 0,04 μl of 50 μM primers couple (Eurogentec), 0,36 μl of DNAse RNAse free water and 1 μl of cDNA matrix (1/50 final dilution). For probes assays, the reaction mixture was prepared in a final volume of 2 μl as follows: 0,4 μl of Real Time Ready DNA Probe Master 5× (Roche), 0,1 μl of Control Setup 20×, 0,1 μl of 4 μM Forward primer (Eurogentec), 0,1 μL of 4 μM Reverse primer (Eurogentec), 0,1 μL of 4 μM FAM/TAMRA Probe (Eurogentec), 0,2 μl of DNAse RNAse free water and 1 μl of cDNA matrix (1/50 final dilution). All pipetting steps were carried out with Agilent Bravo Automated Liquid Handling Platform.

PCR program consists in a first pre-incubation step at 95° C. for 1 min following by 50 PCR cycles (95° C. for 2 sec, 60° C. for 30 sec). Todiscriminate specific from non-specific products and primer dimers, a melting curve was obtained by gradual increase in temperature from 60 to 95° C.

TABLE
Real-Time PCR primers of the 10 Biomarkers
plus the 2 reference genes

| Gene name | Primer foward | Primer reverse |
|---|---|---|
| GAPDH* | ATGGGGAAGGTGA | GGGGTCATTGATGG |
| | AGGTCG | CAACAATA |
| B2M* | GCTCAGTAAAGAC | CATCTGTGGATTCA |
| | ACAACCATCC | GCAAACC |
| ABCC1 | CCAGTGGGGATCG | AGGGGATCATCGAA |
| | GACAGA | GAGGTAAAT |
| ACOX1 | TTTCTTCACTGCA | GGAAAGGAGGGATT |
| | GGGCTTT | TTGAGC |
| HIF1A | TTTTGCTCTTTGT | CCTGGTCCACAGAA |
| | GGTTGGA | GATGTTT |
| IGJ | GGACATAACAGAC | TGGCAATTTCTTAC |
| | TTGGAAGCA | ACTAACCTGA |
| TNFRSF10B | GGTTTCATATTTA | CAAACAAGGAAGCA |
| | ATTTGGTCATGG | CATTGTGTA |
| RPS23 | GATTTGGTCGCAA | TGCCTTTGTATAGG |
| | AGGTCAT | GCCAAA |
| ABCC3 | GGAGGACATTTGG | CCCTCTGAGCACTG |
| | TGGGCTTT | GAAGTC |
| LYN | ATCCAACGTCCAA | AAGGCTACCACAAT |
| | TAAACAGCA | GTCTCCT |
| PARP2 | GGGAAAGGAATCT | TTCTTTAGGCGAGA |
| | ACTTTGCTG | GGCAAA |
| UBE2H | CGCAGGTTTTCCA | ATGGCCATTTCTTC |
| | CTCATCT | CCAAG |

(*housekeeping genes)

The qPCR data were analyzed using the Delta.Ct (ΔCt) method (Livak and Schmittgen, 2001). The ΔCt values were determined for all target genes by subtracting the Ct values from the mean of the Ct values of the two reference genes (housekeeping). The 2 housekeeping genes are B2M (NM_009735, Mus musculus beta-2 microglobulin, mRNA) and GAPDH (NM_002046, glyceraldehyde-3-phosphate dehydrogenase, transcript variant 1, mRNA+NM_001256799 Homo sapiens glyceraldehyde-3-phosphate dehydrogenase, transcript variant 2, mRNA).

### 7. Results

Using the Digital Gene Expression (DGE) method, the transcriptomic profiles of total blood of patients was carried out and 169 genes have been selected with edgeR Method. The analyzed genes have been selected according to (1) mathematic filters with the highest differential Fold Change (>1.5), FDR adjusted p-value criterion (<10%) based on the type I (α=5%) error and (2) biological filters with involvement of targeted genes in specific processes and known metabolic pathways.

In a real time PCR assay, a positive reaction is detected by accumulation of a fluorescent signal. The Ct (cycle threshold) is defined as the number of cycles required for the fluorescent signal to cross the threshold (i.e. exceeds background level). Ct values are inversely proportional to the amount of target nucleic acid in the sample (i.e. the lower the Ct value, the greater the amount of target nucleic acid in the sample).

The clinical phase III study (from AB Science, Id. AB07012) provided samples for an ancillary pharmacogenomic study. RNA blood samples were taken from 119 patients before any treatment and they were analyzed via RT-PCR (reverse transcription polymerase chain reaction). A “genetic fingerprint” was isolated, present in 55.5% of patients, which was highly predictive for overall survival, and furthermore, interacted with the treatment type.

In particular, placebo/gemcitabine-treated patients with the “genetic fingerprint” had the lowest median overall survival (OS) (4.7 months) whereas patients with this “genetic fingerprint” treated with masitinib plus gemcitabine had a median OS of 12.9 months, meaning that OS was increased by 8 months (p-value=0.00000056) (multivariate analysis).

Among the 169 genes, ACOX-1, TNFRSF10B, LYN, HIF1A, UBE2H, PARP2, ABCC1, ABCC3, IGJ and RPS23 genes were selected by the inventors, in agreement with the multi-factorial nature of this indication.

Up to today, no results of treatment of a genetic population in pancreatic cancer patients have been reported. Therefore, the identification of a genetic fingerprint described here opens a new avenue to personalized therapy in this indication.

The genetic fingerprint, based on a specific Delta.Ct (ΔCt) value, can be routinely determined via RT-PCR (reverse transcription polymerase chain reaction) from RNA blood samples. The ΔCt value illustrating the expression level of a given gene in a given patient is obtained from the amplification by RT-PCR of a given gene and after individual normalization with respect to genes of reference (B2M, GAPDH). ΔCt values are inversely proportional to the level of gene expression; therefore, in the case of up-regulated genes a lower ΔCt value indicates a greater level of expression (conversely, the higher the ΔCt value the lower the expression level of the gene), whilst in the case of down-regulated genes a higher ΔCt value indicates a lower level of expression (conversely, the lower the ΔCt value the higher the expression level of the gene).

Patients having a modulated expression pattern in at least one of the 6 following gene combinations eligible for gemcitabine+masitinib treatment:
Combination 1: a ΔCt value for ACOX1<=3.05 and a ΔCt value for TNFRSF10B<=6.1;
Combination 2: a ΔCt value for RPS23>0.35 and a ΔCt value for ACOX1<=3.05,
Combination 3: a ΔCt value for ABCC3<=4.3 and a ΔCt value for LYN<=1.65;
Combination 4: a ΔCt value for HIF1A<=3.95 and a ΔCt value for TNFRSF10<=5.65.
Combination 5: a ΔCt value for ABCC1>3.5 and a ΔCt value for IGJ>7.05.
Combination 6: a ΔCt value for UBE2H>3.7 and a ΔCt value for PARP2>7.1.
Accordingly:
a patient having a ΔCt value for ACOX1 of <=3.05 and TNFRSF10B of <=6.1, predicts a short-term survival if treated with gemcitabine as a single agent and a long-term survival if treated with the combination of gemcitabine and masitinib.
a patient having a ΔCt value for RPS23 of >0.35 and ACOX1 of <=3.05, predicts a short-term survival if treated with gemcitabine as a single agent and a long-term survival if treated with the combination of gemcitabine and masitinib.
a patient having a ΔCt value for ABCC3 of <=4.3 and LYN of <=1.65, predicts a short-term survival if treated with gemcitabine as a single agent and a long-term survival if treated with the combination of gemcitabine and masitinib.
a patient having a ΔCt value for HIF1A of <=3.95 and TNFRSF10B of <=5.65, predicts a short-term survival if treated with gemcitabine as a single agent and a long-term survival if treated with the combination of gemcitabine and masitinib.
a patient having a ΔCt value for ABCC1 of >3.5 and IGJ of >7.05, predicts a short-term survival if treated with gemcitabine as a single agent and a long-term survival if treated with the combination of gemcitabine and masitinib.
a patient having a ΔCt value for UBE2H of >3.7 and PARP2 of >7.1, predicts a short-term survival if treated with gemcitabine as a single agent and a long-term survival if treated with the combination of gemcitabine and masitinib.

### Example 2

### Cross-Validation ACOX1 Gene

ACOX1 is the single most discriminatory factor for masitinib efficacy harboring a hazard ratio of 0.23 (95% CI=[0.10; 0.51]; p-value=0.001). ACOX1 has been cross-validated by a bootstrap method showing that the positive treatment effect obtained in the ACOX1 subgroup was confirmed 567 times out of 1,000 iterations.
The ACOX1 gene has been validated by cross-validation

First, a bootstrap method was used (1,000 iterations) to randomly divide the dataset into a Training set and a Test set in a 1:1 ratio.

Then for each gene, the treatment effect of masitinib with respect to placebo was calculated for the samples P1 (technical duplicate 1), P2 (technical duplicate 2), and P3 (arithmetic mean of samples P1 and P2) and in the following patients' subgroups:

| | | |
|---|---|---|
| Highly over-expressed gene: | DCt ≦ Q1 | N = 30/120 |
| Over-expressed gene: | DCt ≦ median | N = 60/120 |
| Slightly over-expressed gene: | DCt ≦ Q3 | N = 90/120 |
| Slightly under-expressed gene: | DCt > Q1 | N = 90/120 |
| Under-expressed gene: | DCt > median | N = 60/120 |
| Highly under-expressed gene: | DCt > Q3 | N = 30/120 |

A given subgroup is cross-validated if the following three conditions are met:
1. The treatment effect of masitinib is significant and in favor of masitinib in the Training set at an alpha-level of 10%, with a gene expression cut-off defined either by P1, or P2, or P3.
2. The positive treatment effect of masitinib identified in the training set is repeated in the Test set (HR<1) in both samples P1 and P2.
3. The positive treatment effect with masitinib is significant at an alpha-level of 10% in the Test set either in the P1 (N≧15) or the P2 (N≧15) sample.

When breaking down the cross-validations according to the ACOX1 DCt cut-off, the following results were obtained:
444 positive cross-validations out of 1,000 iterations in the subgroup of patients with a highly over-expressed ACOX1 (DCt 5≦Q1).
278 positive cross-validations out of 1,000 iterations in the subgroup of patients with an over-expressed ACOX1 (DCt≦median).
9 positive cross-validations out of 1,000 iterations in the subgroup of patients with a slightly over-expressed ACOX1 (DCt≦Q3).
   With:

Q1=3.02(90% CI=[2.98; 3.09])

Median=3.22(90% CI=[3.15; 3.29])

Q3=3.38(90% CI=[3.30; 3.41])

In conclusion, the ACOX1 DCt cut-off value set at ≦3.05, is a robust value to correlate patients responsive to masitinib treatment and high level of ACOX1 gene expression; reporting a high level of significance (p-value=0.00106673) and strong efficacy estimate (hazard ratio [95% CI]=0.23 [0.10; 0.51]).

1-19. (canceled)

20. An in vitro method for determining the prognosis of pancreatic cancer in a patient, comprising the following steps:
a) measuring the expression level of at least two genes selected from the group comprising: ACOX-1, TNFRSF10B, LYN, HIF1A, UBE2H, PARP2, ABCC1, ABCC3, IGJ and RPS23 or homologous genes, in a blood sample of said patient; and
b) comparing the expression level of said at least two genes to reference values, thereby predicting the life expectancy of said patient suffering from pancreatic cancer.

21. The method according to claim 20, wherein an up-regulated or down-regulated expression level of at least two of the ACOX-1, TNFRSF10B, LYN, HIF1A, UBE2H, PARP2, ABCC1, ABCC3, IGJ and RPS23 genes indicates the life expectancy of said patient.

22. The method according to claim 20, wherein said blood sample is a peripheral whole blood sample.

23. The method according to claim 20, wherein the expression level of a gene is measured as the level of the RNA transcript or the cDNA of said gene.

24. The method according to claim 20, wherein the expression level of a gene is measured as the level of the protein of said gene.

25. The method according to claim 20, wherein the expression level of a gene is measured by real time quantitative PCR performed on the RNA transcript or the cDNA of said gene, to determine the cycle threshold (Ct) value, said value being normalized with respect to the expression level of at least one housekeeping gene to give a value ΔCt, wherein said ΔCt is inversely proportional to the level of the gene expression.

26. The method according to claim 20, wherein the expression level of a gene is measured by real time quantitative PCR performed on the RNA transcript or the cDNA of said gene to determine the cycle threshold (Ct) value, said value being normalized with respect to the expression level of at least one housekeeping gene to give a value ΔCt, wherein the ΔCt is based on the expression level of two housekeeping genes.

27. The method according to claim 20, wherein the expression level of a gene is measured by real time quantitative PCR performed on the RNA transcript or the cDNA of said gene to determine the cycle threshold (Ct) value, said value being normalized with respect to the expression level of at least one housekeeping gene to give a value ΔCt, wherein the ΔCt is based on the expression level of the two housekeeping genes B2M and GAPDH.

28. The method according to claim 20, wherein the expression level of a gene is measured by real time quantitative PCR performed on the RNA transcript or the cDNA of said gene to determine the cycle threshold (Ct) value, said value being normalized with respect to the expression level of at least one housekeeping gene to give a value ΔCt, and wherein said patient has a ΔCt value for ACOX1 less than or equal to 3.05 and TNFRSF10B less than or equal to 6.1.

29. The method according to claim 20, wherein the expression level of a gene is measured by real time quantitative PCR performed on the RNA transcript or the cDNA of said gene to determine the cycle threshold (Ct) value, said value being normalized with respect to the expression level of at least one housekeeping gene to give a value ΔCt, and wherein said patient has a ΔCt value for RPS23 greater than 0.35 and ACOX1 less than or equal to 3.05.

30. The method according to claim 20, wherein the expression level of a gene is measured by real time quantitative PCR performed on the RNA transcript or the cDNA of said gene to determine the cycle threshold (Ct) value, said value being normalized with respect to the expression level of at least one housekeeping gene to give a value ΔCt, and wherein said patient has a ΔCt value for ABCC3 less than or equal to 4.3 and LYN less than or equal to 1.65.

31. The method according to claim 20, wherein the expression level of a gene is measured by real time quantitative PCR performed on the RNA transcript or the cDNA of said gene to determine the cycle threshold (Ct) value, said value being normalized with respect to the expression level of at least one housekeeping gene to give a value ΔCt, and wherein said patient has a ΔCt value for HIF1A less than or equal to 3.95 and TNFRSF10B less than or equal to 5.65.

32. The method according to claim 20, wherein the expression level of a gene is measured by real time quantitative PCR performed on the RNA transcript or the cDNA of said gene to determine the cycle threshold (Ct) value, said value being normalized with respect to the expression level of at least one housekeeping gene to give a value ΔCt, and wherein said patient has a ΔCt value for ABCC1 greater than 3.5 and IGJ greater than 7.05.

33. The method according to claim 20, wherein the expression level of a gene is measured by real time quantitative PCR performed on the RNA transcript or the cDNA of said gene to determine the cycle threshold (Ct) value, said value being normalized with respect to the expression level of at least one housekeeping gene to give a value ΔCt, and wherein said patient has a ΔCt value for UBE2H greater than 3.7 and PARP2 greater than 7.1.

34. An in vitro method for determining the prognosis of pancreatic cancer in a patient, comprising the following steps:
a) measuring the expression level of ACOX-1 or homologous genes in a blood sample of said patient; and
b) comparing the expression level of ACOX-1 or homologous genes to a reference value, thereby predicting the life expectancy of said patient suffering from pancreatic cancer.

35. The method according to claim 34, wherein the expression level of ACOX-1 or homologous genes is measured by real time quantitative PCR performed on the RNA transcript or the cDNA of the gene to determine the cycle threshold (Ct) value, said value being normalized with respect to the expression level of at least one housekeeping gene to give a value ΔCt, and wherein said patient has a ΔCt value for ACOX1 less than or equal to 3.05.

36. The method according to claim 20, wherein said step b) comprises predicting the life expectancy of a patient suffering from pancreatic cancer depending upon the treatment received by said patient.

37. The method according to claim 20, wherein said step b) comprises predicting the life expectancy of a patient suffering from pancreatic cancer depending upon the treatment received by said patient, and wherein said treatment is a gemcitabine-based treatment.

38. The method according to claim 20, wherein said step b) comprises predicting the life expectancy of a patient suffering from pancreatic cancer depending upon the treatment received by said patient, and wherein said treatment comprises administering gemcitabine and masitinib.

39. A kit for determining the prognosis of pancreatic cancer in a patient, comprising means for detecting the level of expression of at least two genes selected from the group comprising ACOX-1, TNFRSF10B, LYN, HIF1A, UBE2H, PARP2, ABCC1, ABCC3, IGJ and RPS23.

40. The method according to claim 34, wherein said step b) comprises predicting the life expectancy of a patient suffering from pancreatic cancer depending upon the treatment received by said patient.

41. The method according to claim 34, wherein said step b) comprises predicting the life expectancy of a patient suffering from pancreatic cancer depending upon the treatment received by said patient, and wherein said treatment is a gemcitabine-based treatment.

42. The method according to claim 34, wherein said step b) comprises predicting the life expectancy of a patient suffering from pancreatic cancer depending upon the treatment received by said patient, and wherein said treatment comprises administering gemcitabine and masitinib.
